(19) United States
(12) Patent Application Publication
(10) Pub. No.: US 2025/0345472 A1
(43) Pub. Date: Nov. 13, 2025

(54) Ultraviolet Light Attachment for Coin Counter

(71) Applicant: Leith Tidwell, Provo, UT (US)

(72) Inventor: Leith Tidwell, Provo, UT (US)

(73) Assignee: Leith Tidwell, Provo, UT (US)

(21) Appl. No.: 18/663,058

(22) Filed: May 13, 2024

(51) Int. Cl. A61L 2/10; A61L 2/26; G07D 9/00

(52) U.S. Cl. CPC A61L 2/10; A61L 2/26; G07D 9/00; A61L 2202/11; A61L 2202/14

(57) ABSTRACT

The present invention comprises a Ultraviolet Light Attachment for Coin Counter designed to reduce and eliminate bacterial and viral pathogens residing on coins. The invention includes an integrated ultraviolet light attachment at the entry point of a coin sorting/counting machine to provide removal of bacteria and viruses on physical currency.

### FIELD OF THE INVENTION

The presented invention relates to the reduction and elimination of bacterial and viral pathogens residing on coins through the use of an ultraviolet light attachment on a coin sorter/counter.

### BACKGROUND

The novel Coronavirus, hereafter COVID-19, presented retailers, restaurants, offices, etc. with a difficult situation as regards to payment reception and collection. State guidance as regards to increasing contactless payment as a method to reduce transmission of COVID-19 resulted in the near elimination of paper currency as a payment option. Insurance and business liability surrounding potential COVID-19 transmission also limited the scope and intent within the payment systems. This left many individuals currently outside the card-oriented banking system with little recourse in regards to obtaining goods and services.

The ultraviolet light spectrum has been scientifically proven to significantly reduce bacteria and viral pathogens. Devices utilizing ultraviolet light to sterilize cellular phones and furniture are currently being marketed to the public. Mass sterilization of paper currency occurs at many stages in the distribution and redistribution of coins throughout the financial system except at the point of sale. This invention allows for a measure of contactless payment at the point of sale using currency instead of the credit system. A need demonstrated by the reduction in paper currency usage during the COVID-19 lockdown and social distancing periods.

### BRIEF SUMMARY OF THE INVENTION

The present invention comprises an Ultraviolet Light Attachment for Coin Counter designed to eliminate bacteria and viruses. The Ultraviolet Light Attachment for Coin Counter is integrated at the point of currency entry and consists of a housing for the ultraviolet light which will be used to eliminate any bacteria or viruses the coins may have collected. Coins are inserted through a coin-sorting slot on the top of the device and pass through an ultraviolet light during the sorting process.

### FIGURE DESCRIPTION

FIG. 1 is an isometric view of an Ultraviolet Light Attachment for Coin Counter with generic elements of a coin counter showing my new design.

FIG. 2 is an orthographic front view thereof.

FIG. 3 is an orthographic side view thereof.

FIG. 4 is a front view showing location of cross-section views thereof.

FIG. 5 is a cross-section view thereof.

FIG. 6 is a cross-section view thereof.

FIG. 7 is a front view showing location of detail views thereof.

FIG. 8 is a detail view thereof.

FIG. 9 is a detail view thereof.

### DETAILED DESCRIPTION OF DRAWINGS

In the following detailed description, the device according to the teachings for this application in the form of an ultraviolet light attachment for coin counter will be described by the embodiments and their function. The ultraviolet light attachment for coin counter is depicted using a circular coin sorter but it's functionality is not limited to that specific coin counter configuration.

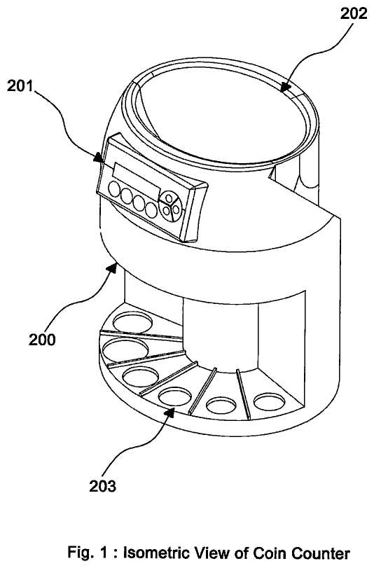

A first embodiment of the ultraviolet light attachment 200 is illustrated in FIG. 1. This view shows the location of the ultraviolet light attachment 200 along with the controlling functional keyboard 201, the coin hopper 202, and the coin channels 203. The coins are inserted into the mechanism through a coin hopper 202 located on top of the coin counter. Coins then move past the ultraviolet light attachment 200 where the bacteria and viruses are removed via the ultraviolet light attachment 200. The coins are then collected in their respective coin channels 203 at the bottom of the device.

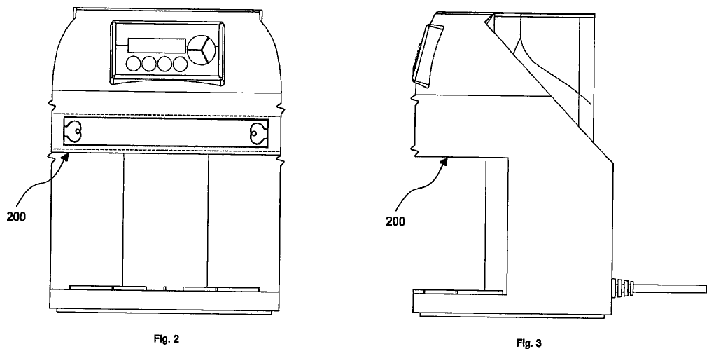

FIG. 2 and FIG. 3 illustrate the location of the ultraviolet light attachment 200 in orthographic side and front views.

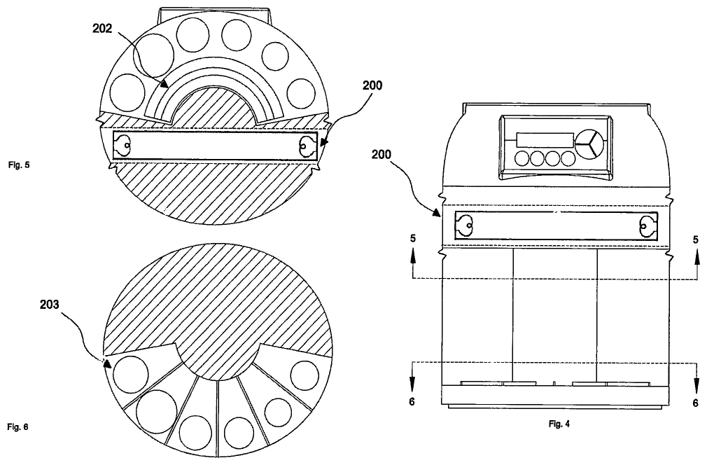

FIG. 4 shows the location of cross-sectional views of the ultraviolet light attachment 200. FIG. 5 illustrates the ultraviolet light attachment 200 below the coin hopper 202. FIG. 6 illustrates the location below the ultraviolet light attachment 200 above the coin channels 203.

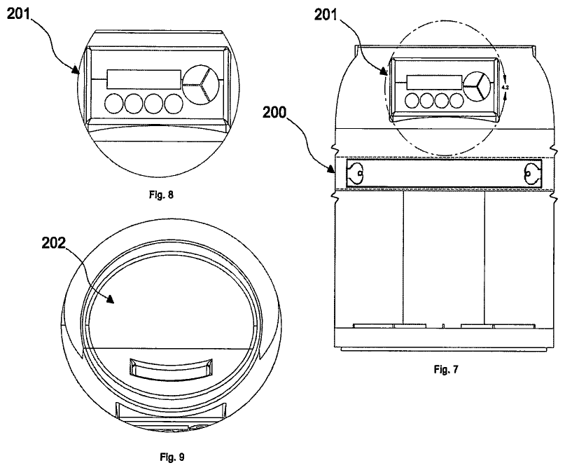

FIG. 7 shows the location of the ultraviolet light attachment 200 in relation to the controlling functional keyboard 201 and coin hopper 202. FIG. 8 is a detail view of the controlling functional keyboard 201. FIG. 9 is a detail view of the coin hopper 202 above the ultraviolet light attachment 200. The ultraviolet light attachment 200 will be controlled by use of functional keyboard module 201. Module will include functionality for coin counter as well as ultraviolet light attachment 200. All coins will enter device through coin hopper 202 located on top of coin counter and will pass through ultraviolet light attachment 200 during counting process.

### Line Description

Even dashed lines show placement of cross-sectional views. Dashed-dot lines show boundary of detail views. Zigzag lines indicate boundary of claimed invention.

### Broken Line and Boundary Line Statement

The dashed-dot lines illustrate boundary lines and form no part of the claimed design. The even dashed lines represent unclaimed subject matter and form no part of the claimed design. The claimed design is bounded by broken zigzag lines and the elements located above and below zigzag lines form no part of claimed design.

1. I claim the design of an Ultraviolet Light Attachment for Coin Counter as shown.
